(19) United States
(12) Patent Application Publication
(10) Pub. No.: US 2024/0189490 A1
(43) Pub. Date: Jun. 13, 2024

(54) ANTIMICROBIAL ELUTING AIRWAY DEVICES

(71) Applicant: THE CHILDREN'S HOSPITAL OF PHILADELPHIA, Philadelphia, PA (US)

(72) Inventors: Riccardo L. GOTTARDI, Philadelphia, PA (US); Matthew R. ARONSON, Philadelphia, PA (US)

(73) Assignee: THE CHILDREN'S HOSPITAL OF PHILADELPHIA, (US)

(21) Appl. No.: 18/553,893

(22) PCT Filed: Apr. 8, 2022
(86) PCT No.: PCT/US22/24050

§ 371 Date: Oct. 4, 2023

Related U.S. Application Data
(60) Provisional application No. 63/172,177, filed on Apr. 8, 2021.

(51) Int. Cl. A61L 31/16; A61L 31/04; A61L 31/06; A61M 16/04

(52) U.S. Cl. CPC A61L 31/16; A61L 31/048; A61L 31/06; A61M 16/0402; A61L 2300/252; A61L 2300/404; A61L 2420/02; A61M 2205/0205; A61M 2205/0238; A61M 2207/00

(57) ABSTRACT

The present disclosure provides improved airway intervention devices that are coated with a material and contain an antimicrobial peptide. The devices are useful in controlling the microbial flora of the airway and preventing infections and conditions such as subglottic stenosis, pneumonia (VAP), laryngeal infection, post-operative dressing/packing-induced infection, upper airway infection, rhinosinusitis, choanal atresia, vocal fold injury and paralysis.

### PRIORITY CLAIM

This application claims benefit of priority to U.S. Provisional Application Ser. No. 63/172,177, filed Apr. 8, 2021, the entire contents of which are hereby incorporated by reference.

### FEDERAL FUNDING STATEMENT

The invention was made with government support under grant no. 7229370000 awarded by the National Science Foundation. The government has certain rights in the invention.

### BACKGROUND

### 1. Field

The disclosure relates generally to the field of medicine, medical devices and biologics. More particularly, it concerns compositions and devices for delivery antimicrobial agents to a subject's airway through eluting airway intervention devices.

### 2. Related Art

Endotracheal tubes (ETT) are widely used for intubations in the hospital in emergency situations, surgeries, and chronically ventilated patients. As ETTs remain in the airway of patients for time durations as long as two weeks, there is a growing problem of bacterial adherence to the tube producing harmful biofilms, as well as the overgrowth of certain pathogenic bacteria in the airway microbiome. One particular example is subglottic stenosis (SGS), characterized by narrowing of the laryngeal airway starting at the vocal folds and extending to the lower border of the cricoid cartilage. This is caused by dysregulated laryngotracheal fibroblasts leading to the formation of scar tissue.¹⁻³ Mild grades of SGS can often be treated with balloon dilation or endoscopic interventions.⁴ In contrast, severe SGS often requires open laryngotracheal reconstruction or resection.⁵ Unfortunately, most treatments have high rates of restenosis, indicating a clinical need for alternative or preventative therapies. Recent studies have shown correlations between SGS and dysregulated laryngotracheal microbiomes as indicated by an overabundance of Mycobacterium species,⁶ inverse correlations between the genera Streptococcus and commensal Prevotella,⁷ as well as associations between pathogens within the genera Moraxella and Acinetobacter.⁷ Therefore, improved methods of microbial modulation in SGS is currently in great need.

### SUMMARY

In a first embodiment, the present disclosure provides an airway intervention device where a surface of said device is coated with a material comprising one or more anti-microbial peptides (AMPs) and one or more hydrophobic and/or natural polymers. The device may be a tracheal tube, a stent, a mask, a tracheostomy tube, a catheter, an oral retainer, a balloon, a patch, or a packing material. The polymer may poly-(lactic-co-glycolic acid), poly glycolic acid, poly lactic acid, poly (lactic-co-glycolic acid), or any combination thereof, or poly caprolactone, hydrogel, alginate, polyurethane, polyester, poly (ethylene terephthalate), poly anhydrides, poly orthoesters, poly beta-amino esters, chitosan, hyaluronic acid, cellulose, collagen, gelatin, silk fibroin, and/or cyclodextrin.

The one or more AMPs may be an anionic peptide, a linear cationic α-helical peptide, a cationic peptide enriched in one or more of proline, arginine, phenylalanine, glycine or tryptophan, or a anionic/cationic peptide containing cysteine with at least one intrapeptide disulfide bond, and optionally may contain one or more feature selected from a β-hairpin structure, a cyclic peptide structure, enrichment in any natural amino acid, a noncanonical amino acid, self-assembly, or D, L or D/L enantiomeric amino acids. The anionic peptide may be a dermicidin or Maximin H5. The linear cationic α-helical peptide may be a halictine, a citropin, an aurein, a temporin, a macropin, a cecropin, an andropin, a moricin, a ceratoxin, a melittin, a magainin, a dermaseptin, a bominin, brevinin-1, an esculentin, buforin II, a lassioglossin, CAP18 or LL37. The cationic peptide enriched in one or more of proline, arginine, phenylalanine, glycine or tryptophan may be an abaecin, a drosocin, an apidaecin, a diptericin, an attacin, a prophenin, or an indolicidin. The anionic/cationic peptide containing cysteine with at least one intrapeptide disulfide bond may be a brevenin, a protegrin, a tachyplesin, a defensin or a drosomycin.

The coated material may permit controlled release of said AMP, such as 1 ng/day to 200 μg/day. The material may further comprise an anti-inflammatory agent, such as a steroid or an NSAID, or an additional molecule that impair a bacterial or viral agent, such as a protein that interferes with pathogen attachment, colonization, a protein that enhances immune clearance of said agent, or a conventional antibiotic, such as azithromycin, tobramycin, ciprofloxacin, erythromycin, and amoxicillin. The anti-inflammatory agent or bacterial/viral impairing agent may be formulated for controlled/delayed release.

In another embodiment, there is provided a method of preparing a coated airway intervention device comprising (a) providing an airway intervention device; (b) immersing said device in an emulsion comprising one or more hydrophobic or natural polymers and one or more anti-microbial peptides (AMPs) to coat said device; (c) repeating step (b) at least once, optionally twice; and (d) drying said coated device. The device may be a tracheal tube, a stent, a mask, a tracheostomy tube, a catheter, an oral retainer, a balloon, a patch, or a packing material. The polymer may poly-(lactic-co-glycolic acid), poly glycolic acid, poly lactic acid, poly (lactic-co-glycolic acid), or any combination thereof, or poly caprolactone, hydrogel, alginate, polyurethane, polyester, poly (ethylene terephthalate), poly anhydrides, poly orthoesters, poly beta-amino esters, chitosan, hyaluronic acid, cellulose, collagen, gelatin, silk fibroin, and/or cyclodextrin.

The one or more AMPs may be an anionic peptide, a linear cationic α-helical peptide, a cationic peptide enriched in one or more of proline, arginine, phenylalanine, glycine or tryptophan, or a anionic/cationic peptide containing cysteine with at least one intrapeptide disulfide bond, and optionally may contain one or more feature selected from a β-hairpin structure, a cyclic peptide structure, enrichment in any natural amino acid, a noncanonical amino acid, self-assembly, or D, L or D/L enantiomeric amino acids. The anionic peptide may be a dermicidin or Maximin H5. The linear cationic α-helical peptide may be a halictine, a citropin, an aurein, a temporin, a macropin, a cecropin, an andropin, a moricin, a ceratoxin, a melittin, a magainin, a dermaseptin, a bominin, brevinin-1, an esculentin, buforin II, a lassioglossin, CAP18 or LL37. The cationic peptide enriched in one or more of proline, arginine, phenylalanine, glycine or tryptophan may be an abaecin, a drosocin, an apidaecin, a diptericin, an attacin, a prophenin, or an indolicidin. The anionic/cationic peptide containing cysteine with at least one intrapeptide disulfide bond may be a brevenin, a protegrin, a tachyplesin, a defensin or a drosomycin.

The coated material may permit controlled release of said AMP, such as 1 ng/day to 200 μg/day. The material may further comprise an anti-inflammatory agent, such as a steroid or an NSAID, or an additional molecule that impair a bacterial or viral agent, such as a protein that interferes with pathogen attachment, colonization, a protein that enhances immune clearance of said agent, or a conventional antibiotic, such as azithromycin, tobramycin, ciprofloxacin, erythromycin, and amoxicillin. The anti-inflammatory agent or bacterial/viral impairing agent may be formulated for controlled/delayed release.

In yet another embodiment, there is provided a method of providing airway intervention to a subject comprising inserting an airway intervention device as described herein. The intervention may be performed for less than 1 day, or more than 1 day, such as for a week, two weeks, three weeks, four weeks, five weeks, six weeks, seven weeks, eight weeks, nine weeks or 10 weeks.

In further embodiment, there is provided a kit comprising a coated airway intervention device as described herein, or the individual components for making such a coated airway intervention device, in sterile packaging. The kit may further comprise instructions for use of said device.

In yet a further embodiment, there is provided a method of reducing the incidence of subglottic stenosis, pneumonia (VAP), laryngeal infection, post-operative dressing/packing-induced infection, upper airway infection, rhinosinusitis, choanal atresia, or vocal fold injury and paralysis in subjects receiving airway intervention comprising providing airway intervention with an airway intervention device as described herein.

It is contemplated that any method or composition described herein can be implemented with respect to any other method or composition described herein. For example, a compound synthesized by one method may be used in the preparation of a final compound according to a different method.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Schematic illustration of peptide/PLGA coated endotracheal tubes. When placed in physiologically relevant environments, the PLGA undergoes hydration and bulk erosion as it degrades and subsequently elutes the antimicrobial peptide from the tube. The free peptide then interacts with bacteria in the upper airway to elicit antimicrobial effects.

FIGS. 2A-C. Surface and release characterization of FITC-BSA coated ET Tubes. (FIG. 2A) Fluorescent micrographs and (FIG. 2B) total encapsulation of increasing dip repetitions (n=5), and (FIG. 2C) cumulative release profile of FITC-BSA (green, n=8) and PLGA degradation (black, n=2) over time. Scale bars=200 μm.

FIGS. 3A-E. Scanning electron microscopy of (FIG. 3A) PLGA only and (FIG. 3B) peptide/PLGA coated endotracheal (ET) tubes showing improved surface uniformity with peptide/PLGA coatings. Cross sectional micrographs of (FIG. 3C) PLGA only and (FIG. 3D) peptide/PLGA coated tubes showing differences in orthogonal surface smoothness. (FIG. 3E) Cumulative release profile of peptide/PLGA (n=2) coated ET tubes over time. Scale bars=25 μm.

FIGS. 4A-C. Antibacterial activity of drug-eluting endotracheal tubes against S. epidermidis in (FIG. 4A) preventing planktonic growth in broth (n≥12), (FIG. 4B) reducing biofilm growth (n≥14), and (FIG. 4C) inhibiting viable bacterial adherence (n≥8). Uncoated (−) is no coating, PLGA is only PLGA coating, 0.5×MIC/PLGA is 0.5×MIC peptide with PLGA coatings, and 1×MIC/PLGA is 1×MIC peptide with PLGA coatings, and free peptide (+) is peptide dissolved in medium. * p<0.05, ** p<0.01, *** p<0.001, **** p<0.0001.

FIGS. 5A-C. Peptide coated ET Tubes and controls treated against (FIG. 5A) Streptococcus pneumoniae (n=10) and (FIG. 5B) patient derived oral microbiomes (n=4). (FIG. 5C) Biocompatibility toward laryngotracheal fibroblasts (n=8). Uncoated (−) is no coating, PLGA is only PLGA coating, 0.5×MIC/PLGA is 0.5×MIC peptide with PLGA coatings, and 1×MIC/PLGA is 1×MIC peptide with PLGA coatings, and free peptide (+) is peptide dissolved in medium. * p<0.05, ** p<0.01, *** p<0.001, **** p<0.0001.

FIG. 6. Schematic of peptide coated endotracheal tubes to elicit antimicrobial effects. Tubes are first coated in a PLGA+peptide emulsion before subsequently undergoing controlled release in physiological environmental conditions. The peptide then disrupts bacterial cell membranes to kill pathogenic microbes and prevent biofilm formation.

FIGS. 7A-C. (FIG. 7A) Percent viability of S. epidermidis when treated with peptide coated tubes and controls. (FIG. 7B) Percent viability of S. epidermidis against different concentrations of peptide loaded tubes and controls. (FIG. 7C) Colony forming units on endotracheal tubes after 24 hours of incubation with S. epidermidis. ** p<0.01 **** p<0.0001.

FIG. 8. A schematic illustration of coated endotracheal tube platform inside of the upper airway. On the right, there are three false-colored scanning electron microscopy images of the different tube conditions after treatment with the bacteria S. epidermidis for 24 hours. The top image is a blank, uncoated tube and there are many bacteria (pink) present on the tube (yellow) surface. The middle picture is a tube coated with only PLGA (purple), and there are bacteria (pink) present along with biofilm production (green). The bottom picture is a tube coated with PLGA and antimicrobial peptide (purple) on the tube (yellow) with all bacterial growth inhibited. Images of the coated tubes are displayed as insets.

FIG. 9. The tube platform was re-engineered to fit a mouse trachea (1 mm diameter) for future in vivo testing. Tubes were also prepared with A4K14-citropin 1.1 instead of Lasioglossin-III demonstrating the ability to include any peptide successfully in the platform with robust delivery and activity.

FIG. 10. To optimize the tubes for mouse tracheal size, a higher concentration of peptide was required to be coated on the tube. In this experiment, the inventors used a fluorescent protein (FITC-BSA) to determine the ability to quadruple the loading. When the volume of aqueous phase (containing the protein) was changed and increased by 4, the amount that was loaded onto the tube after dipping also increased by 4. This indicates that the amount of peptide loaded on the tube is finely tunable.

FIG. 11. Profile of FITC-BSA (model protein) and citropin antimicrobial peptide (active agent) releasing from the tube. On this mouse sized tube, the inventors loaded 17.04 μg of Citropin peptide onto the tube, which released linearly at a rate of 0.397 μg/day over 9 days. This is the normal time frame that patients receive a new endotracheal tube, so it is ideal that it is releasing ˜80% of the encapsulated peptide by day 9. Additionally, a linear release is preferred such that there is a continuous supply of peptide to the region of interest and it is not cleared from the body too rapidly to take effect.

FIG. 12. (Left) The inventors cultured the different mouse size tube conditions against the bacteria S. epidermidis and saw significant reductions in bacterial viability with peptide was loaded onto the tube (**** p<0.0001). (Right) The inventors then removed the tracheal complexes from mice, inserted tubes, and cultured them in vitro for 24 hours. This experiment is considered ex vivo since the inventors are culturing them in the trachea of a mouse, but in a dish instead of the live animal. The results show that there are significant reductions in the number of bacteria on the tube (colony forming units) with the Peptide/PLGA coated tubes compared to the PLGA only or uncoated tubes (**** p<0.0001).

FIG. 13. Fluorescent microscopy images of PLGA/FITC-BSA coated endotracheal tubes modifying PLGA and PVA concentration, as well as dip repetition. Main image scale bar=1 mm; inset scale bar −200 μm.

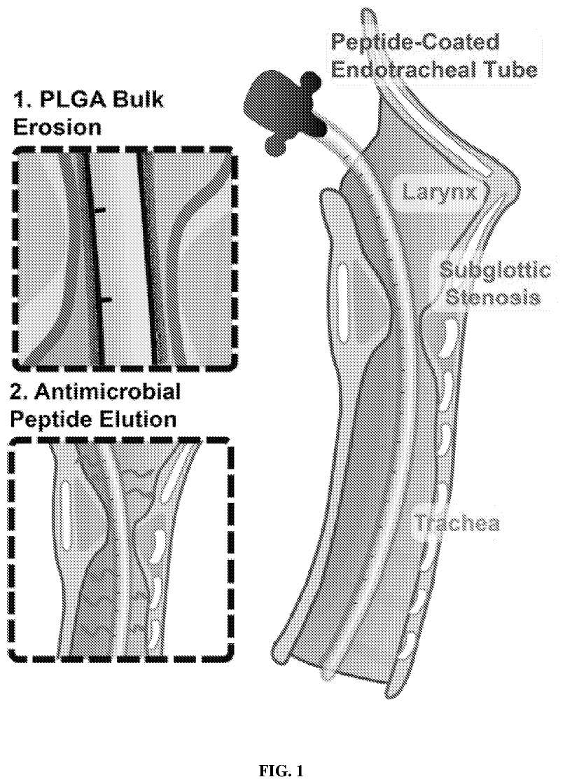

### DESCRIPTION OF ILLUSTRATIVE EMBODIMENTS

As discussed above, bacterial damage during airway intervention therapy is a major problem. One possible approach to this challenge would be through controlled drug delivery that uses biomaterials composed of either natural or synthetic polymers that deliver therapeutic and/or diagnostic molecules to a target area of interest. Once localized to the desired area, the biomaterial delivers the cargo through a variety of means. Commonly with device coatings, the biomaterial degrades over time when implanted in physiological conditions where the matrix slowly and temporally releases the cargo.

The present inventors chose to encapsulate naturally derived antimicrobial peptides (AMPs) in a polymeric poly(lactic-co-glycolic acid) (PLGA) to coat endotracheal tubes (ETTs) to modulate the microbiome of the airway. AMPs offer numerous advantages over other antibacterial compounds due to their high biocompatibility, selectivity to bacterial cells over healthy human cells, and selectivity toward specific bacteria. Furthermore, they undergo temporal degradation which ensures the therapeutic is eliminated from the system locally and avoids collateral damage to healthy cells and other organ systems.

The inventors' goal was to form a homogeneous antimicrobial coating on ETTs for the prevention of adhering bacteria and overgrowing pathogenic species in the airway during acute and chronic intubation. This technology will, in one aspect, be deployed to assist in the preventative treatment of subglottic stenosis (SGS). SGS is the narrowing of the airway below the vocal folds because of fibrotic scarring after intubation, which causes breathing and speech difficulties. Current therapies include systemic anti-inflammatory medications or invasive procedures to surgically enlarge the airway lumen; however, both approaches present a high risk of restenosis. Therefore, alternative local delivery therapies are required to more effectively manage this disease.

Although the etiology of SGS is yet to be fully elucidated, recent work has highlighted a relationship with upper airway microbiome imbalances. Therefore, the inventors engineered a drug-eluting endotracheal tube (ETT) to deliver an antimicrobial therapy for the local modulation of the microbiome during intubation. They demonstrated how a homogeneous coating of the ETTs with the antimicrobial peptide Lasioglossin-III in a PLGA matrix yields a prolonged, linear release over two weeks, retains antimicrobial activity, and is cytocompatible toward human fibroblasts.

This same platform technology approach can be applied to controllably release any other AMP loaded into the engineered drug-eluting coating for the selective killing of pathogenic bacteria. Additionally, the formulation offers the option to include other combinatorial treatments such as anti-inflammatory drugs and conventional antibiotics, that could be released simultaneously or asynchronously in a controlled manner. For instance, a peptide and an anti-inflammatory therapy combination could be both incorporated in the same coating polymeric matrix for dually loaded action. The organization of the coating can be arranged to separately control the release profile over time of peptide and of the anti-inflammatory agent, combining for instance a rapid release of the anti-inflammatory agent and the delayed and continuous release of the AMP, or any other temporal combination. Examples of additional agents this technology could be applied to include the recently discovered ACE2 mimicking peptide that binds tightly to the spike protein on SARS-COV-2. This peptide could be used to coat endotracheal tubes, nasal airway stents, or other airway implants to inhibit the SARS-COV-2 from entering cells and infecting patients.

Furthermore, the technology is not limited to just ETTs, but other airway intervention devices as well, such as stents or that could be coated in the same manner to achieve modulation of the microbiome of the airway and local modulation of the inflammatory response. Additionally, the material could be used to coat masks to prevent the pathogens from entering the body outside of medical settings.

### I. Airway Intervention Devices

Airway management is a critical component of healthcare. This includes anything from simply clearing the airway to managing complex aspiration issues using specialized equipment. In general, airway management equipment is often categorized as (1) facemask ventilation devices, which may use additional attachments; (2) supraglottic airway devices; (3) tracheal intubation; (4) suction machines for airway clearance; and (5) transtracheal access.

### A. Tracheal Tubes

A tracheal tube is a catheter that is inserted into the trachea for the primary purpose of establishing and maintaining a patent airway and to ensure the adequate exchange of oxygen and carbon dioxide. Many different types of tracheal tubes are available, suited for different specific applications:
endotracheal tubes are a specific type of tracheal tube that is nearly always inserted through the mouth (orotracheal) or nose (nasotracheal).
tracheostomy tubes are another type of tracheal tube; this 2-3-inch-long (51-76 mm) curved metal or plastic tube may be inserted into a tracheostomy stoma (following a tracheotomy) to maintain a patent lumen.
tracheal buttons are rigid plastic cannula about 1 inch in length that can be placed into the tracheostomy after removal of a tracheostomy tube to maintain patency of the lumen.

Most endotracheal tubes today are constructed of polyvinyl chloride, but specialty tubes constructed of silicone rubber, latex rubber, or stainless steel are also widely available. Most tubes have an inflatable cuff to seal the trachea and bronchial tree against air leakage and aspiration of gastric contents, blood, secretions, and other fluids. Uncuffed tubes are also available, though their use is limited mostly to pediatric patients (in small children, the cricoid cartilage, the narrowest portion of the pediatric airway, often provides an adequate seal for mechanical ventilation).

Types of endotracheal tubes include oral or nasal, cuffed or uncuffed, preformed (e.g., RAE (Ring, Adair, and Elwyn) tube), reinforced tubes, and double-lumen endobronchial tubes. For human use, tubes range in size from 2 to 10.5 mm in internal diameter (ID). The size is chosen based on the patient's body size, with the smaller sizes being used for pediatric and neonatal patients. Tubes larger than 6 mm ID usually have an inflatable cuff. Originally made from red rubber, most modern tubes are made from polyvinyl chloride. Those placed in a laser field may be flexometallic. Robertshaw (and others) developed double-lumen endo-bronchial tubes for thoracic surgery. These allow single-lung ventilation while the other lung is collapsed to make surgery easier. The deflated lung is re-inflated as surgery finishes to check for fistulas (tears). Another type of endotracheal tube has a small second lumen opening above the inflatable cuff, which can be used for suction of the nasopharngeal area and above the cuff to aid extubation (removal). This allows suctioning of secretions that sit above the cuff which helps reduce the risk of chest infections in long-term intubated patients.

So called “armored” endotracheal tubes are cuffed, wire-reinforced, silicone rubber tubes that are quite flexible but yet difficult to compress or kink. This can make them useful for situations in which the trachea is anticipated to remain intubated for a prolonged duration, or if the neck is to remain flexed during surgery. Polyvinyl chloride tubes are relatively stiff in comparison. Preformed tubes (such as the oral and nasal RAE tubes, named after the inventors Ring, Adair and Elwyn) are also widely available for special applications. These may also be constructed of polyvinyl chloride or wire-reinforced silicone rubber. Other tubes (such as the Bivona Fome-Cuf tube) are designed specifically for use in laser surgery in and around the airway. Various types of double-lumen endotracheal (actually, endobronchial) tubes have been developed (Carlens, White, Robertshaw, etc.) for ventilating each lung independently—this is useful during pulmonary and other thoracic operations.

### B. Stents

Airway stents, also known as tracheobronchial prostheses, are tube-shaped devices with a hollow lumen that are inserted into an airway. They are usually placed bronchoscopically and can be used to treat a variety of large airway diseases. Airway stenting is, in general, a palliative therapy or bridge to curative therapy for patients with several types of airway diseases, among which central airway obstruction (CAO) due to malignancy is the most common.

### C. Airway Masks

Face mask ventilation is a vital tool providing support to patients in a number of different contexts. This intervention is often appropriate for patients showing symptoms of hypoxic respiratory failure, apnea, an inability to protect the airway, or an altered mental state caused by exertion or hypoxia. The masks may or may not have features that extend into the mouth and/or trachea of the subject.

### D. Supraglottic Airway Devices

Supraglottic airway devices target the upper airway. These devices open the upper airway to allow for unobstructed ventilation. They may also replace some other airway management devices. According to research published in 2014, supraglottic airway devices successfully provide rescue ventilation in more than 90 percent of patients for whom tracheal intubation and mask ventilation are impossible. Though effective, these devices may also increase the risk of airway damage and pulmonary aspiration.

Supraglottic airways (SGAs) are a group of airway devices that can be inserted into the pharynx to allow ventilation, oxygenation, and administration of anesthetic gases, without the need for endotracheal intubation. Supraglottic airway devices (SADs) are used to keep the upper airway open to provide unobstructed ventilation. Early (first-generation) SADs rapidly replaced endotracheal intubation and face masks in >40% of general anesthesia cases due to their versatility and ease of use.

A laryngeal mask airway (LMA), also known as laryngeal mask, is a particular type of SGA device that keeps a patient's airway open during anesthesia or unconsciousness. It is a type of supraglottic airway device. A laryngeal mask is composed of an airway tube that connects to an elliptical mask with a cuff which is inserted through the patient's mouth, down the windpipe, and once deployed forms an airtight seal on top the glottis (unlike tracheal tubes which pass through the glottis) allowing a secure airway to be managed by a health care provider.

LMAs are most commonly used by anesthetists to channel oxygen or anaesthesia gas to a patient's lungs during surgery and in the pre-hospital setting (for instance by paramedics and emergency medical technicians) for unconscious patients.

A laryngeal mask has an airway tube that connects to an elliptical mask with a cuff. The cuff can either be of the inflating type (achieved after insertion using a syringe of air), or self-sealing. Once inserted correctly (and the cuff inflated where relevant) the mask conforms to the anatomy with the bowl of the mask facing the space between the vocal cords. After correct insertion, the tip of the laryngeal mask sits in the throat against the muscular valve that is located at the upper portion of the esophagus.

### E. Other Devices

Other devices that may be used in airway intervention include catheters, oral retainers, balloons, patches, or a packing material. The surface of any of these devices may also be coated with the materials disclosed herein.

### II. Antimicrobial Peptides and Other Agents

### A. Antimicrobial Peptides

Antimicrobial peptides (AMPs), also called host defense peptides (HDPs) are part of the innate immune response found among all classes of life. Fundamental differences exist between prokaryotic and eukaryotic cells that may represent targets for antimicrobial peptides. These peptides are potent, broad spectrum antibiotics which demonstrate potential as novel therapeutic agents. Antimicrobial peptides have been demonstrated to kill Gram-negative and Gram-positive bacteria, enveloped viruses, fungi and even transformed or cancerous cells. Unlike the majority of conventional antibiotics, it appears that antimicrobial peptides frequently destabilize biological membranes, can form transmembrane channels, and may also have the ability to enhance immunity by functioning as immunomodulators.

Antimicrobial peptides are a unique and diverse group of molecules, which are divided into subgroups on the basis of their amino acid composition and structure. Antimicrobial peptides are generally between 12 and 50 amino acids. These peptides include two or more positively charged residues provided by arginine, lysine or, in acidic environments, histidine, and a large proportion (generally >50%) of hydrophobic residues. The secondary structures of these molecules follow 4 themes, including i) α-helical, ii) β-stranded due to the presence of 2 or more disulfide bonds, iii) ß-hairpin or loop due to the presence of a single disulfide bond and/or cyclization of the peptide chain, and iv) extended. Many of these peptides are unstructured in free solution, and fold into their final configuration upon partitioning into biological membranes. It contains hydrophilic amino acid residues aligned along one side and hydrophobic amino acid residues aligned along the opposite side of a helical molecule. This amphipathicity of the antimicrobial peptides allows them to partition into the membrane lipid bilayer. The ability to associate with membranes is a definitive feature of antimicrobial peptides, although membrane permeabilization is not necessary. These peptides have a variety of antimicrobial activities ranging from membrane permeabilization to action on a range of cytoplasmic targets.

TABLE A
Antimicrobial Peptides

| Type | characteristic | AMPs |
|---|---|---|
| Anionic peptides | rich in glutamic | Maximin H5 from |
| | and aspartic acids | amphibians. Dermicidin |
| | | from humans |
| Linear cationic | lack in cysteine | Cecropins, andropin, |
| α-helical peptides | | moricin, ceratotoxin and |
| | | melittin from insects, |
| | | Magainin, dermaseptin, |
| | | [symbol] , brevinin-1, |
| | | esculentins and buforin |
| | | II from amphibians, |
| | | CAP18 from rabbits, |
| | | LL37 from humans |
| Catonic peptide | rich in proline, | abaecin and drosocin, |
| enriched for | arginine, | apidaecin, diptericin, |
| specific amino | phenylalanine, | and attacin from insects, |
| acid | glycine, tryptophan | prophenin from pigs, |
| | | indolicidin from cattle. |
| Anionic and | contain 1~3 | 1 bond:brevinins, 2 |
| cationic peptides | disulfide bond | bonds:protegrin from pig, |
| that contain | | tachyplesins from |
| cysteine and form | | horseshoe crabs, 3 bonds: |
| disulfide bonds | | defensins from humans, |
| | | more than 3:drosomycin |
| | | in fruit flies |

[symbol]

indicates data missing or illegible when filed

The modes of action by which antimicrobial peptides kill microbes are varied and may differ for different bacterial species. Some antimicrobial peptides kill both bacteria and fungi, e.g., psoriasin kills E. coli and several filamentous fungi. The cytoplasmic membrane is a frequent target, but peptides may also interfere with DNA and protein synthesis, protein folding, and cell wall synthesis. The initial contact between the peptide and the target organism is electrostatic, as most bacterial surfaces are anionic, or hydrophobic, such as in the antimicrobial peptide piscidin. Their amino acid composition, amphipathicity, cationic charge and size allow them to attach to and insert into membrane bilayers to form pores by ‘barrel-stave’, ‘carpet’ or ‘toroidal-pore’ mechanisms. Alternately, they may penetrate into the cell to bind intracellular molecules which are crucial to cell living. Intracellular binding models includes inhibition of cell wall synthesis, alteration of the cytoplasmic membrane, activation of autolysin, inhibition of DNA, RNA, and protein synthesis, and inhibition of certain enzymes. However, in many cases, the exact mechanism of killing is not known. One emerging technique for the study of such mechanisms is dual polarisation interferometry. In contrast to many conventional antibiotics these peptides appear to be bactericidal instead of bacteriostatic. In general, the antimicrobial activity of these peptides is determined by measuring the minimal inhibitory concentration (MIC), which is the lowest concentration of drug that inhibits bacterial growth.

AMPs can possess multiple activities including anti-gram-positive bacterial, anti-gram-negative bacterial, anti-fungal, anti-viral, anti-parasitic, and anti-cancer activities. A big AMP functional analysis indicates that among all AMP activities, amphipathicity and charge, two major properties of AMPs, best distinguish between AMPs with and without anti-gram-negative bacterial activities. This implies that being AMPs with anti-gram-negative bacterial activities may prefer or even require strong amphipathicity and net positive charge.

In addition to killing bacteria directly they have been demonstrated to have a number of immunomodulatory functions that may be involved in the clearance of infection, including the ability to alter host gene expression, act as chemokines and/or induce chemokine production, inhibiting lipopolysaccharide induced pro-inflammatory cytokine production, promoting wound healing, and modulating the responses of dendritic cells and cells of the adaptive immune response. Animal models indicate that host defense peptides are crucial for both prevention and clearance of infection. It appears as though many peptides initially isolated as and termed “antimicrobial peptides” have been shown to have more significant alternative functions in vivo (e.g., hepcidin). Dusquetide for example is an immunomodulator that acts through p62, a protein involved in toll like receptor based signalling of infection. The peptide is being examined in a Phase III clinical trial by Soligenix (SGNX) to ascertain if it can assist in repair of radiation-induced damage to oral mucosa arising during cancer radiotherapy of the head and neck.

Antimicrobial peptides possessing a net positive charge are attracted and incorporated into negatively charged bacterial membranes. Once inside the membrane, they are believed to cause disruption through three possible mechanisms: (a) toroidal pore formation; (b) carpet formation; and (c) barrel stave formation. Although the specifics of each mechanism differ, all propose peptide-induced membrane rupture, allowing cytoplasmic leakage that ultimately leads to death.

Recent work has painted a more complex picture of antimicrobial peptide activity. Antimicrobial peptides may also function as metabolic inhibitors, inhibitors of DNA, RNA, and protein synthesis, and inhibitors of cell wall synthesis or septum formation. They are also known to cause ribosomal aggregation and delocalize membrane proteins. Adding a further layer of complexity, many natural antimicrobial peptides possess weak bactericidal activity. Rather than directly inhibit bacterial growth, they are now known to act in concert with the host immune system through mechanisms including chemokine induction, histamine release, and angiogenesis modulation. These immunomodulatory effects have only recently begun to receive attention.

Several methods have been used to determine the mechanisms of antimicrobial peptide activity. In particular, solid-state NMR studies have provided an atomic-level resolution explanation of membrane disruption by antimicrobial peptides In more recent years, X-ray crystallography has been used to delineate in atomic detail how the family of plant defensins rupture membranes by identifying key phospholipids in the cell membranes of the pathogen. Human defensins have been thought to act through a similar mechanism, targeting cell membrane lipids as part of their function. In fact, human beta-defensin 2 have now been shown to kill the pathogenic fungi Candida albicans through interactions with specific phospholipids. From the computational point of view, the molecular dynamics simulations can shed light in the molecular mechanism and the specific peptide interactions with lipids, ions and solvent.

In the competition of bacterial cells and host cells with the antimicrobial peptides, antimicrobial peptides will preferentially interact with the bacterial cell to the mammalian cells, which enables them to kill microorganisms without being significantly toxic to mammalian cells. Selectivity is a very important feature of the antimicrobial peptides and it can guarantee their function as antibiotics in host defense systems.

There are some factors that are closely related to the selectivity property of antimicrobial peptides, among which the cationic property contributes most. Since the surface of the bacterial membranes is more negatively charged than mammalian cells, antimicrobial peptides will show different affinities towards the bacterial membranes and mammalian cell membranes.

In addition, there are also other factors that will affect the selectivity. It's well known that cholesterol is normally widely distributed in the mammalian cell membranes as a membrane stabilizing agents but absent in bacterial cell membranes; and the presence of these cholesterols will also generally reduce the activities of the antimicrobial peptides, due either to stabilization of the lipid bilayer or to interactions between cholesterol and the peptide. So the cholesterol in mammalian cells will protect the cells from attack by the antimicrobial peptides.

Besides, the transmembrane potential is well known to affect peptide-lipid interactions. There is an inside-negative transmembrane potential existing from the outer leaflet to the inner leaflet of the cell membranes and this inside-negative transmembrane potential will facilitate membrane permeabilization probably by facilitating the insertion of positively charged peptides into membranes. By comparison, the transmembrane potential of bacterial cells is more negative than that of normal mammalian cells, so bacterial membrane will be prone to be attacked by the positively charged antimicrobial peptides. Similarly, it is also believed that increasing ionic strength, which in general reduces the activity of most antimicrobial peptides, contributes partially to the selectivity of the antimicrobial peptides by weakening the electrostatic interactions required for the initial interaction.

The cell membranes of bacteria are rich in acidic phospholipids, such as phosphatidylglycerol and cardiolipin. These phospholipid headgroups are heavily negatively charged. Therefore, the outmost leaflets of the bilayer which is exposed to the outside of the bacterial membranes are more attractive to the attack of the positively charged antimicrobial peptides. Thus, the interaction between the positive charges of antimicrobial peptides and the negatively charged bacterial membranes is mainly the electrostatic interactions, which is the major driving force for cellular association. In addition, since antimicrobial peptides form structures with a positively charged face as well as a hydrophobic face, there are also some hydrophobic interactions between the hydrophobic regions of the antimicrobial peptides and the zwitterionic phospholipids (electrically neutral) surface of the bacterial membranes, which act only as a minor effect in this case.

In contrast, the outer part of the membranes of plants and mammals is mainly composed of lipids without any net charges since most of the lipids with negatively charged headgroups are principally sequestered into the inner leaflet of the plasma membranes. Thus, in the case of mammalian cells, the outer surfaces of the membranes are usually made of zwitterionic phosphatidylcholine and sphingomyelin, even though a small portion of the membrane's outer surfaces contain some negatively charged gangliosides. Therefore, the hydrophobic interaction between the hydrophobic face of amphipathic antimicrobial peptides and the zwitterionic phospholipids on the cell surface of mammalian cell membranes plays a major role in the formation of peptide-cell binding. However, the hydrophobic interaction is relatively weak when compared to the electrostatic interaction, thus, the antimicrobial peptides will preferentially interact with bacterial membranes.

Much effort has been put into controlling cell selectivity. For example, attempts have been made to modify and optimize the physicochemical parameters of the peptides to control the selectivities, including net charge, helicity, hydrophobicity per residue (H), hydrophobic moment (u) and the angle subtended by the positively charged polar helix face (@). Other mechanisms like the introduction of D-amino acids and fluorinated amino acids in the hydrophobic phase are believed to break the secondary structure and thus reduce hydrophobic interaction with mammalian cells. It has also been found that Pro→Nlys substitution in Pro-containing β-turn antimicrobial peptides was a promising strategy for the design of new small bacterial cell-selective antimicrobial peptides with intracellular mechanisms of action. It has been suggested that direct attachment of magainin to the substrate surface decreased nonspecific cell binding and led to improved detection limit for bacterial cells such as Salmonella and E. coli.

Bacteria use various resistance strategies to avoid antimicrobial peptide killing. Some microorganisms alter net surface charges. Staphylococcus aureus transports D-alanine from the cytoplasm to the surface teichoic acid which reduces the net negative charge by introducing basic amino groups. S. aureus also modifies its anionic membranes via MprF with L-lysine, increasing the positive net charge. The interaction of antimicrobial peptides with membrane targets can be limited by capsule polysaccharide of Klebsiella pneumoniae. Alterations occur in Lipid A. Salmonella species reduce the fluidity of their outer membrane by increasing hydrophobic interactions between an increased number of Lipid A acyl tails by adding myristate to Lipid A with 2-hydroxymyristate and forming hepta-acylated Lipid A by adding palmitate. The increased hydrophobic moment is thought to retard or abolish antimicrobial peptide insertion and pore formation. The residues undergo alteration in membrane proteins. In some Gram-negative bacteria, alteration in the production of outer membrane proteins correlates with resistance to killing by antimicrobial peptides. Non-typeable Hemophilus influenzae transports AMPs into the interior of the cell, where they are degraded. Furthermore, H. influenzae remodels its membranes to make it appear as if the bacterium has already been successfully attacked by AMPs, protecting it from being attacked by more AMPs. ATP-binding cassette transporters import antimicrobial peptides and the resistance-nodulation cell-division efflux pump exports antimicrobial peptides. Both transporters have been associated with antimicrobial peptide resistance. Bacteria produce proteolytic enzymes, which may degrade antimicrobial peptides leading to their resistance.^{[55]} Outer membrane vesicles produced by Gram-negative bacteria bind the antimicrobial peptides and sequester them away from the cells, thereby protecting the cells. The outer membrane vesicles are also known to contain various proteases, peptidases and other lytic enzymes, which may have a role in degrading the extracellular peptide and nucleic acid molecules, which if allowed to reach to the bacterial cells may be dangerous for the cells. Cyclic-di-GMP signaling had also been involved in the regulation of antimicrobial peptide resistance in Pseudomonas aeruginosa.

### B. Other Agents

The coating may also contain other active agents besides the AMPs described above. For example, anti-inflammatory agents such as steroids or NSAIDS may be included to reduce inflammation and the resulting tissue damage. In addition, other agents that impair the growth, replication and/or colonization of bacterial, fungal or viral pathogens maybe included, such as conventional antibiotics. An example of an additional agent is the ACE2 mimicking peptide that binds tightly to the spike protein on SARS-COV-2. When included in the coating of the present disclosure, this peptide could inhibit the SARS-COV-2 from entering cells and infecting patients. Another example would be an antibody that targets the infectious agent and that optionally may carrier an effector molecule (drug).

### III. Polymers and Methods of Coating

A. Hydrophobic and/or Natural Polymers

A polymer is a substance or material consisting of very large molecules or macromolecules composed of many repeating subunits. Due to their broad spectrum of properties, both synthetic and natural polymers play essential and ubiquitous roles in everyday life. Polymers range from familiar synthetic plastics such as polystyrene to natural biopolymers such as DNA and proteins that are fundamental to biological structure and function. Polymers, both natural and synthetic, are created via polymerization of many small molecules, known as monomers. Their consequently large molecular mass, relative to small molecule compounds, produces unique physical properties.

Hydrophobic polymers may be amphiphilic in nature in that they have regions that are more hydrophobic in nature and regions that are more hydrophilic in nature. The relative extent of hydrophobicity and hydrophilicity will determine the solubility of the material. In practice, the materials that are, overall, more hydrophobic will be relatively more water insoluble and oil soluble. Conversely, the more hydrophilic materials will be relatively more water soluble and oil insoluble.

Polymers may be copolymers. The term copolymer is understood to be a polymeric molecule that contains two or more different monomers. They can be copolymers of more than one monomer or polymers where a fraction of the monomers are chemically derivatized. Materials having utility in the present invention are known in the art by a number of terms including, but not limited to, hydrophobic polymers, amphiphilic polymers, hydrophobically modified polymers, and polymeric surfactants. These materials can be made by a number of processes, including, but not limited to, synthesis of polymers from one or more monomers, derivatization of existing polymer or grafting; isolation from a natural source. One approach to producing a hydrophobic polymer is by direct polymerization of monomers, which includes a least one hydrophobic monomer, to form a copolymer. Polymerization may be done by any method known in the art, including solution, dispersion and inverse emulsion polymerization.

### B. Coating Methods

In general, the coating methods will rely on treating a device surface multiple times with the coating material and the AMP. This is advantageously performed by applying an emulsion of the coating material and AMP, such as by painting, spraying, dipping or other exposing the device to the emulsion, optionally repeating this process to produce multiple layers or an deepened single layer that is both smooth and capable of continued/prolonged release (e.g., in an aqueous environment) of the AMP over time.

Depending on the type of polymer and its molecular weight, the thickness and shape of the coating will vary, in particular the bubbles in which the AMP is entrapped. This will material impact the release profile and can be tailored to treating particular conditions as well as to control specific pathogens.

An exemplary protocol for coating an endotracheal tube (ET) is as follows. ETs were coated by first forming a polymer and protein emulsion, followed by the dipping of ET tubes. All tubes were first cut into 1 cm cylindrical segments. Next, a water (w) in oil (o) emulsion was prepared. 50 μL of 8 mg/mL peptide in 1% PVA (w) was combined with 1 mL of 1% PLGA in DCM (o). The solution was then subjected to 20 seconds of a 40 Joule sonic dismembrator at 25% amplitude. Peptide ET tubes were dipped thrice 10 seconds each, with 20 seconds at room temperature before dips. Coated ET tubes were then lyophilized overnight and stored in a desiccator at room temperature until use. The coatings are uniform and smooth on the surface, and between 500 nm and 200 μm thick.

The rate of degradation of the polymer is between 0.1 mg and 200 μg per day. The rate of peptide release is between 1 ng and 200 μg per day. The polymer in the coating degrades when placed in aqueous environments by hydrolysis allowing for the elution of the peptide. The molecular weight of the polymer dictates the rate of degradation, which in combination with the coating shape and thickness and with the concentration and size (from 1 micron to less than 1 nm) of the emulsified bubbles dictates the rate of release of the therapeutic peptide from the coating.

AMPs can act as surfactants because they are amphipathic and can stabilize the emulsion in which they are applied to the device. This can decrease the size of the encapsulated bubbles/pockets of AMP offering another element of control of the release profile. Furthermore, this helps achieved the desired smoothness of the coating surface. Peptides being amphipathic, can also associate directly/form a complex with the hydrophobic polymer during fabrication rather than remaining in separate bubbles/pockets. As such, their release would then be directly linked to the polymer hydrolyzation and be less dependent on the size of the pockets.

### IV. Kits

In various aspects of the embodiments, a kit is envisioned containing the devices (coated or uncoated) and other agents including hydrophobic polymers and AMPs. In some embodiments, the disclosure contemplates a kit for preparing and/or employing an airway intervention device. The kit may comprise one or more sealed vials containing any of the pharmaceutical compositions of the present embodiments. The kit may include, for example, polymers, AMPs, as well as reagents to prepare, formulate, and/or apply the emulsions of polymers and AMPs. In some embodiments, the kit may also comprise a suitable container, which is a container that will not react with components of the kit. The container may be made from sterilizable materials so that the device remains uncontaminated during shipping and storage.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering an airway intervention or preparing a device for using in the same.

### V. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Chemical and Biological Materials. Urea, arginine, and crystal violet were purchased from Acros Organics. Vitamin K was purchased from Alpha Aesar. Agar, peptone, KCl, K2HP04, sucrose, BD Difeo Nutrient Broth, BD Bacto Tryptic Soy Broth, and BD Bacto Brain Heart Infusion were purchased from Fisher Scientific. 4 and 5 mm Shiley oral/nasal cuffless, murphy eye, non-DEHP ET tubes were purchased from Medline. Dichloromethane (DCM), PLGA (50:50, 7-17 kDa), poly(vinyl alcohol) (PVA), albumin-fluorescein isothiocyanate conjugate (FITC-BSA), NaOH, NaCl, Na₂HPO₄, HEPES, hemin chloride, N-acetylmuramic acid, and porcine mucin type III were purchased from Sigma Aldrich. Fetal bovine serum (FBS), Antibiotic-Antimycotic (Anti-Anti), Fluorobrite Dulbecco's Modified Eagle Medium (DMEM), minimum essential amino acids (MEM NEAA), Bacto Yeast Extract, and Bacto Tryptone were purchased from ThermoFisher Scientific. Patient derived laryngotracheal fibroblasts were obtained from pediatric donors with signed informed consent, and approval from the institutional review board (Children's Hospital of Philadelphia IRB #19-016327). Pooled oral microbiome samples were obtained from adult donor saliva samples with signed informed consent, and approval from the institutional review board (Children's Hospital of Philadelphia IRB #20-018262).

Dip Coating of Endotracheal Tubes. ET tubes were coated by first forming a polymer and protein emulsion, followed by the dipping of ET tubes. 5 mm ET tubes were used for FITC-BSA coatings, and 4 mm ET tubes were used for peptide coatings. All tubes were first cut into 1 cm cylindrical segments. Next, a water (w) in oil (o) emulsion was prepared as described previously.²⁵ In brief, 50 μL of 20 mg/mL FITC-BSA in 1% PVA (w) was combined with 1 mL of 1% PLGA in DCM (o). Peptide emulsions were prepared similarly, except 8 mg/mL peptide in the place of FITC-BSA. The solution was then subjected to 20 seconds of a 40 Joule sonic dismembrator (Fisher Scientific) at 25% amplitude. FITC-BSA ET tubes were dipped in the emulsion once, twice, or thrice for 10 seconds with 20 seconds at room temperature between each dip; peptide ET tubes were only dipped thrice. Coated ET tubes were then lyophilized overnight and stored in a desiccator at room temperature until use.

Surface Characterization. FITC-BSA coated ET tubes were first cut in smaller parts and subsequently imaged using a BZ-X810 All-in-One Fluorescent Microscope with a GFP filter (Keyence). Peptide coated ET tubes were cut into four quadrants to evaluate the surface and into hnm cylinders to evaluate the cross section. Imaging was conducted via scanning electron microscopy with a FEI Quanta 600 FEG Mark II ESEM with a 5 kV accelerating voltage.

Release Quantification. Release of loaded protein or peptide from ET tubes was achieved from quartered tube segments in PBS at 37° C. while shaking at 100 rpm. FITC-BSA elution and PLGA degradation samples were obtained by completely removing the supernatant and adding fresh PBS to each well. FITC-BSA release was measured via fluorescence on a Synergy H1 Microplate Reader (BioTek) with 485 nm excitation and 515 nm emission wavelengths. PLGA degradation was quantified via EnzyFluo L-Lactate Assay Kit (Bioassay Systems). Peptide release was quantified via Pierce Quantitative Fluorometric Peptide Assay (Thermo Scientific). Total encapsulation of FITC-BSA coated tubes was determined by 2.5M NaOH degradation at room temperature rotating at 40 rpm for 72 hours and measuring fluorescence as previously described.

Bacteria Culture. Staphylococcus epidermidis (Winslow and Winslow) Evans (ATCC 13990) was cultured in nutrient broth aerobically at 37° C. Streptococcus pneumoniae (Klein) Chester (NCTC 7465; ATCC 33400) was cultured in Brain Heart Infusion Broth and plated on Trypticase soy agar with 5% defibrinated sheep blood (Colorado Serum Co.) aerobically (5% CO₂) at 37° C.

Oral Microbiome Isolation and Culture. Oral microbiome cultures were created and propagated as previously described with slight modification.³⁶ In brief, 2 mL of saliva was collected from 4 individuals and centrifuged at 200 rcf to pellet mammalian cells and debris. Supernatants were pooled and 1 mL was used to inoculate 5 mL of pre-reduced SHI medium. Samples were cultured anaerobically (85% N₂, 10% CO₂, 5% H₂) at 37° C. After 24 hours of incubation, cells were centrifuged at 2600 rcf, resuspended in pre-H₂) at 37° C. After 24 hours of incubation, cells were centrifuged at 2600 rcf, resuspended in pre-20% glycerol and stored at −80° C. 1 mL of frozen culture was added to 5 mL pre-reduced SHI medium and left for 24 hours prior to experimentation.

Antibacterial Activity Assay. Minimum inhibitory concentrations (MIC), defined as the lowest amount of peptide required to inhibit all growth of bacteria, of Lasio against S. epidermidis and S. pneumoniae were achieved as previously described.²⁷ Antibacterial activity of ET tubes were initiated by submerging peptide/PLGA coated tubes and control tubes (uncoated tubes, PLGA only coated tubes, free peptide) in 500 μL of diluted bacteria (OD600=0.001), and incubated for 24 hours at 37° C. shaking at 100 rpm. For quantifying bacterial growth in broth, 100 μL of bacterial supernatant was removed and OD600 was measured. For quantifying biofilm inhibition, tubes were first rinsed in PBS, incubated with 0.5% crystal violet for 10 minutes, washed with PBS to remove excess crystal violet, and dried overnight. Then, crystal violet was dissolved in 95% ethanol and quantified by absorbance at 595 nm by plate reader. Adherent bacterial viability was quantified by removing adherent bacteria via sonication of tubes in 750 μL of filtered HEPES/Saline (70 mM NaCl, 0.75 mM Na₂PO₄, 25 mM HEPES), and subsequently diluted and plated on appropriate agar plates. After 24 hours of incubation, colonies were counted by ImageJ software.

Biocompatibility Toward Laryngotracheal Fibroblasts. Peptide/PLGA coated tubes and controls were first submerged in Fluorobrite DMEM supplemented with 10% FBS, 2% Anti-Anti, 1% MEM NEAA, and incubated for 24 hours at 37° ° C. while shaking at 100 rpm to achieve release. Simultaneously, laryngotracheal fibroblasts were seeded at 7,000 cells/well in 48 well plates and allowed to adhere overnight in a culture atmosphere at 37° C., 95% relative humidity, and 5% CO₂. Cell media was replaced with appropriate tube releasate and incubated for 24 hours in the culture atmosphere. Invitrogen AlamarBlue HS Cell Viability Reagent (ThermoFisher Scientific) was used to quantify viability.

Statistical Methods. All data are graphed as means±standard deviation and reported as means with 95% confidence intervals (CI). For antibacterial activity analysis, outliers were removed using a two-sided Grubbs' test with α=0.01. Ordinary one-way ANOVA with Tukey's multiple comparisons was utilized unless standard deviations were statistically different, in which case, Brown-Forsythe and Welch ANOVA tests with Dunnett T3 multiple comparisons were conducted. Data analysis were performed using GraphPad Prism Software Version 9.0.2.

### Example 2—Results

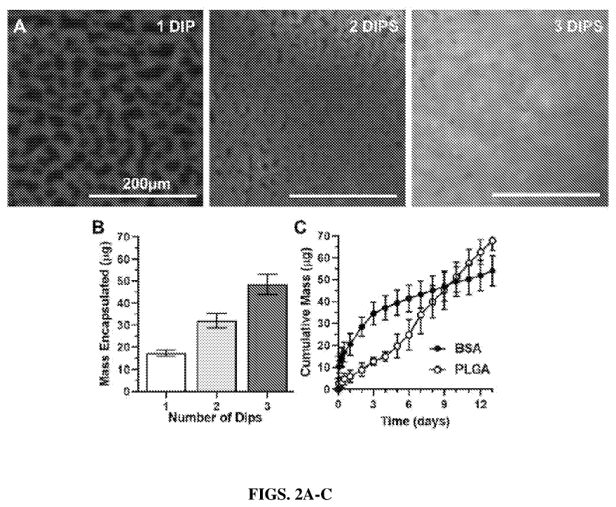

Fluorescent BSA Coated ET Tubes Demonstrate Coating Feasibility. PLGA (1 and 5%) and PV A (0.1 and 1%) concentrations were varied in FITC-BSA coated ET tubes to determine optimal polymer to surfactant ratio that yields a smooth coating. Fluorescent microscopy revealed improved surface homogeneity with 1% PLGA and 1% PVA coatings (FIG. 13). Additionally, different dip repetitions were tested (once, twice, thrice) to improve coating uniformity, revealing the most uniform coating after three dips (FIG. 2A). Notably, each dip added a reproducible coating of approximately 17 μg (n=5, 95% CI: 15.71-18.97) (FIG. 2B). When placed in PBS at 37° ° C. shaking at 100 rpm, the release profile of FITC-BSA exhibited two sequential release phases, an initial burst release from 0-3 days and a linear release (1.89 μg/day, n=8, 95% CI: 1.80-1.99) (FIG. 2C, green). This correlates to a similar PLGA (FIG. 2C, black) degradation profile providing evidence that following the initial burst release and hydration of the coating, protein diffusion from the tube is linearly dependent on polymer degradation.

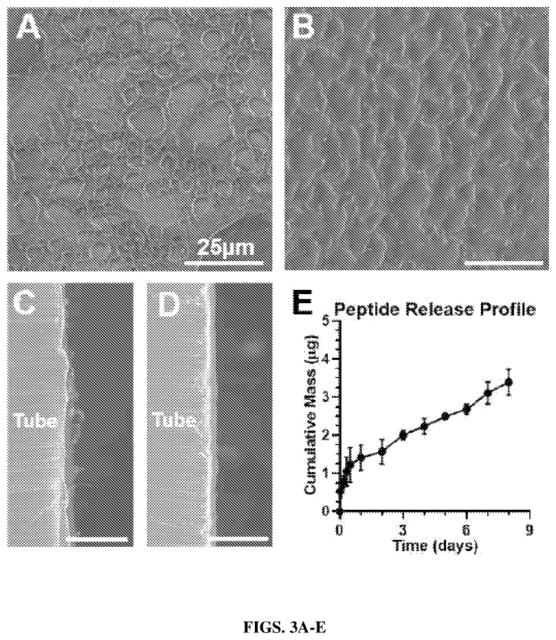

Peptide Coated ET Tubes Exhibit Uniform Coating and Release. Scanning electron microscopy (SEM) was conducted on PLGA and peptide/PLGA coated tubes to determine surface characterization. PLGA coated tubes revealed a non-uniform surface coating (FIG. 3A), while the peptide/PLGA coatings were homogenous and smooth (FIG. 3B). Furthermore, cross-sectional imaging of orthogonal roughness revealed decreased surface perturbations with peptide/PLGA compared to PLGA coatings (FIG. 3C-D). The release profile of Lasio from peptide/PLGA tubes demonstrated a burst release for the first day, followed by linear release (0.28 μg/day, n=2, 95% CI: 0.26-0.31).

Peptide Coated ET Tubes Inhibit Bacterial Growth and Adherence. ET tube constructs were used against Staphylococcus epidermidis, a gram-positive bacterium, to determine the ability of the different tube coating conditions to (1) inhibit planktonic bacterial growth in liquid medium, (2) prevent bacterial adherence, and (3) reduce adherent bacterial viability. 1×MIC/PLGA peptide coatings (n=19) exhibited significant bacterial inhibition compared to uncoated (n=19), PLGA coated (n=11), and 0.5×MIC/PLGA peptide (n=12) coated tubes (p<0.0001) (FIG. 4A). Adherent bacterial populations were quantified with a crystal violet biofilm assay revealing similar abundances between 1×MIC/PLGA peptide coatings (n=16) and uncoated (n=16), PLGA coated (n=16), 0.5×MIC/PLGA peptide (n=14) coatings (FIG. 4B). There was, however, a significant reduction in adherent bacteria between 1×MIC/PLGA peptide coatings (n=16) and the free peptide control (n=18) (p<0.0001). The viability of adherent bacteria was significantly reduced with 1×MIC/PLGA peptide coated tubes compared to uncoated, PLGA coated, and 0.5×MIC/PLGA peptide coatings (n=10, p<0.0001); furthermore, free peptide (n=8) inhibited the viability of adherent bacteria compared to 1×MIC/PLGA peptide coatings (p=0.0015) (FIG. 4C).

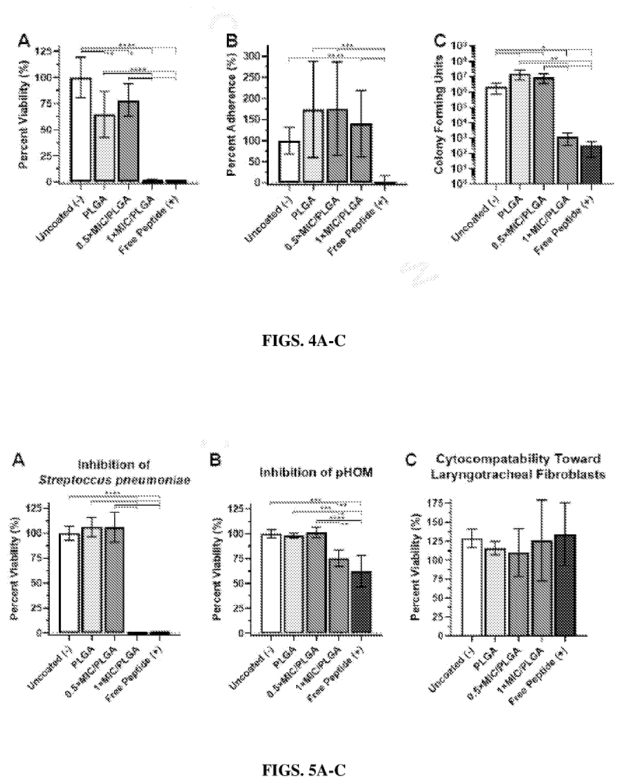

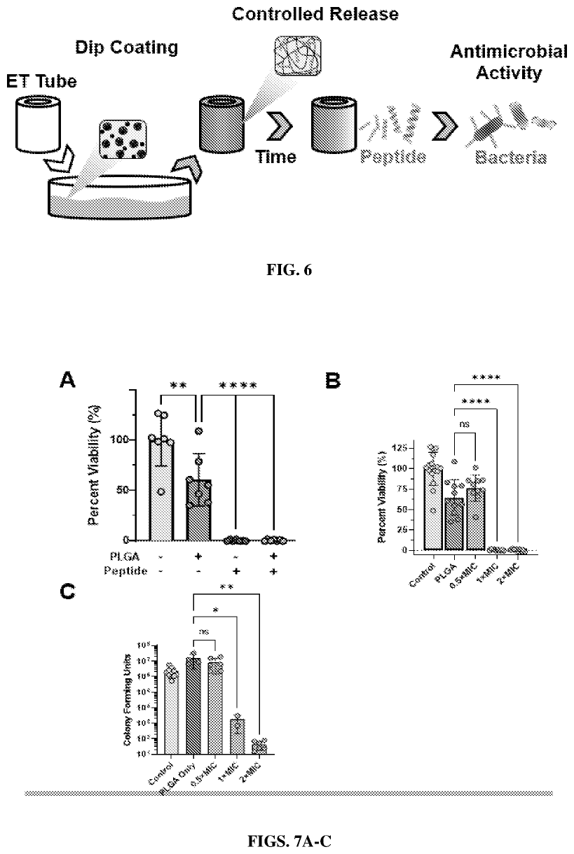

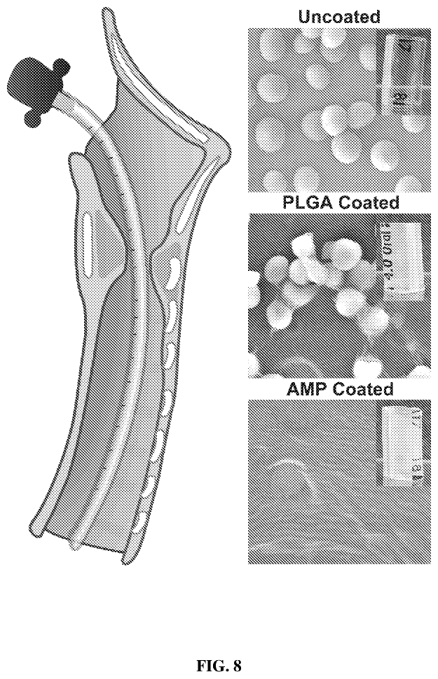

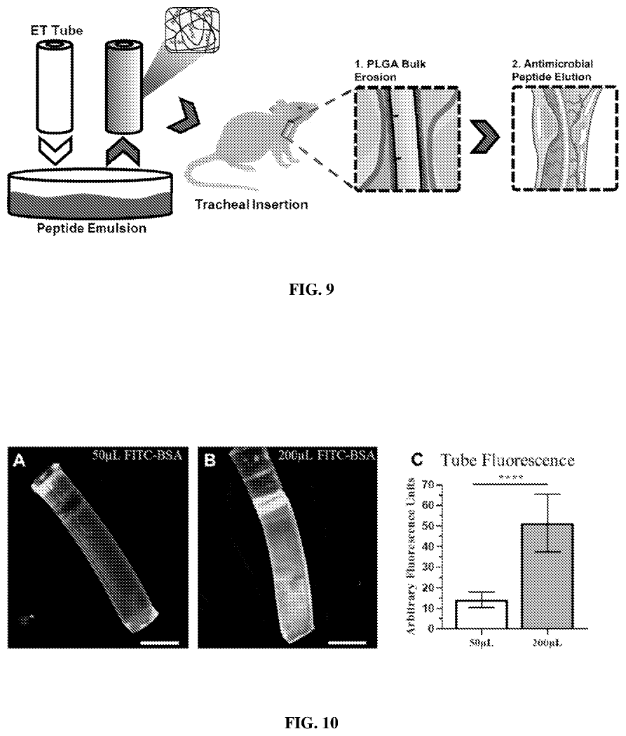

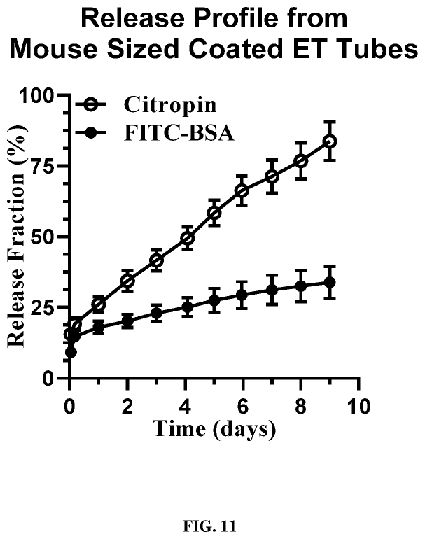

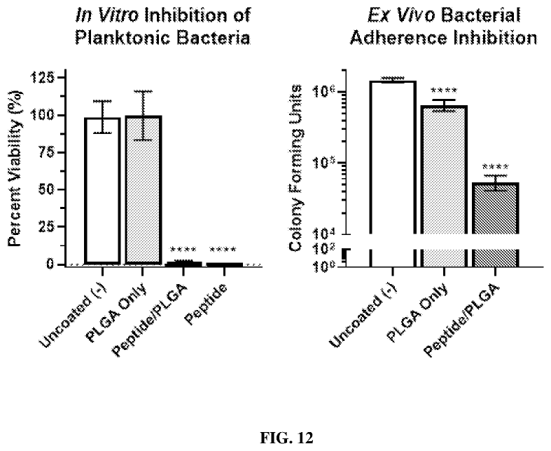

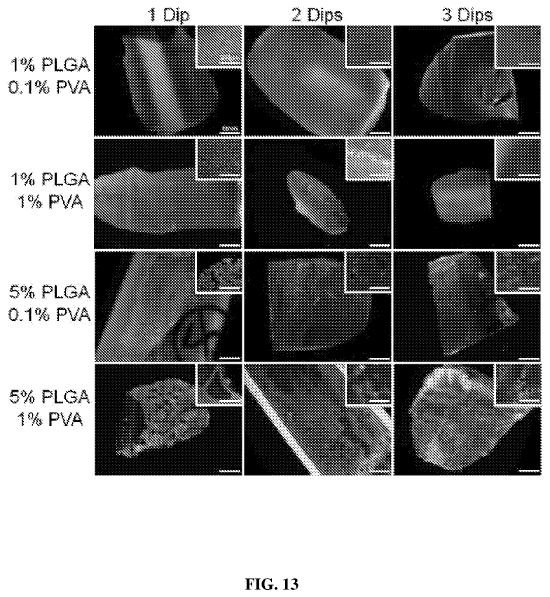

Peptide Coated ET Tubes Inhibit Airway Pathogens. Peptide/PLGA coated ET tubes and controls were used to treat the Gram-positive microbe Streptococcus pneumoniae, showing significant inhibition of bacterial growth in broth with 1×MIC/PLGA peptide (n=10) compared to uncoated (n=10), PLGA coated (n=10), and 0.5×MIC/PLGA peptide (n=12) coated tubes (p<0.0001) (FIG. 5A). The same tube conditions were used to treat pooled human oral microbiome (pHOM) communities demonstrating significant differences in overall bacterial viability with 1×MIC/PLGA peptide coatings (75.47%, 95% CI: 62.41-88.54, p<0.05) and free peptide (62.34%, 95% CI: 37.00-87.68, p<0.001) compared to uncoated (100%, 95% CI: 93.74-106.3), PLGA coated (94.46%, 95% CI: 94.46-102.1), and 0.5×MIC/PLGA peptide (101.4%, 95% CI: 93.19-109.7) coated tubes (FIG. 5B).

Peptide Coated ET Tubes Are Biocompatible with Laryngotracheal Fibroblasts. Patient derived laryngotracheal fibroblasts were treated with peptide coated ET tubes and controls (n=8). All treatments exhibited viability of >100%; notably, 125% with 1×MIC/PLGA peptide coated ET tubes (95% CI: 81.14-170.50) suggesting fibroblast viability and proliferation is unhindered by the tube releasate (FIG. 5C).

### Example 3—Discussion

In this study, the inventors developed a platform approach to rapidly and uniformly coat ET tubes with the model antimicrobial peptide Lasioglossin-III, which elutes linearly and exhibits antimicrobial activity to modulate the upper-airway microbiome and reduce bacterial inflammation in the evolution of SGS. An effective antimicrobial eluting coating must meet two key requirements: surface smoothness that would avoid airway irritation and limit microbial adhesion, and a continuous release over one to two weeks, the typical timeframe that results in ET tube biofilm formation.^{28.29} PLGA was selected as the polymer matrix for coating based on its biocompatibility, previous use in FDA approved devices, and controllable degradation.^{30.31} The inventors then used the model fluorescent protein FITC-BSA to optimize the PLGA/PV A ratio (1% PLGA, 1% PVA) and number of dips to obtain a uniform and smooth coating on the ET tube and an effective release profile (FIG. 2A, FIG. 13). Dipping thrice resulted in a smooth, reproducible coating (FIG. 2B) and, as the polymer progressively degrades, in a continuous linear release of protein over two weeks (FIG. 2C).

The inventors then created an ET tube coating that would release Lasioglossin-111, which the inventors chose as a model AMP for its broad spectrum antibacterial activity.²⁴ Coating characterization by SEM revealed improved surface homogeneity (FIGS. 3A-B) and cross-sectional smoothness (FIGS. 3C-D) of Lasio/PLGA coatings compared to PLGA alone. This is not unexpected, since like most AMPs Lasio is amphipathic²⁷ and can stabilize the coating emulsion, improving uniformity. As predicted by the inventors' model, the release of Lasio from coated ET tubes rapidly reaches the expected effective concentration and is continuous and linear for over one week (FIG. 3E). Importantly, these findings suggest that most AMPs would result in a stable, uniform coating, and in a predictable, linear release. Hence, this platform technology could be easily applied to a wide range of peptides that could have broad or targeted antimicrobial properties, depending on the desired target.

The inventors also showed that the Lasio eluting ET tube is effective against a model gram-positive airway microbe S. epidermidis.³² Since the minimum inhibitory concentration of Lasio against S. epidermidis was determined to be 1.25 M (data not shown), the inventors engineered a Lasio/PLGA coating that would release 1×MIC in 24 hours. These results showed that planktonic bacterial growth in medium was significantly reduced by the 1×MIC/PLGA peptide coated tubes compared to tubes that were uncoated, coated only with PLGA, and coated with only 0.5×MIC/PLGA peptide (p<0.0001) (FIG. 4A). Clinically, this highlights the ability for Lasio to elute from the tube during intubation and kill bacteria present in the trachea mucus. Although bacterial adhesion to 1×MIC/PLGA peptide coated tubes was not significantly different compared to all other conditions, the viability of adherent bacteria was significantly reduced for the 1×MIC/PLGA coated tubes by over 1,500-fold compared to all other conditions (p<0.0001) (FIG. 4C). Thus, the inventors successfully showed that Lasio/PLGA coated ET tubes can both inhibit planktonic bacterial growth and reduce biofilm viability potentially reducing the risk for infection in intubated patients.

Finally, the inventors tested the efficacy of this peptide eluting ET tube against the upper-airway respiratory pathogen S. pneumoniae (pneumococcus) and on pooled human oral microbiome samples. Pneumococcus is of specific interest because, besides causing pneumonia and sinus infections,³³ Streptococcus species are more abundant on endotracheal tubes and in SGS patients.^{7.34} The inventors demonstrated that 1×MIC/PLGA peptide coated ET tubes significantly reduced the planktonic growth of S. pneumoniae compared to all other conditions (p<0.0001), as would occur in the tracheal mucosa! layer of an intubated patient (FIG. 5A). In parallel, the coated tubes also reduced the viability of pHOM showing the broad-spectrum antibiotic activity of the Lasio/PLGA coating toward a polymicrobial community (FIG. 5B). This confirms that the broad spectrum antimicrobial activity of Lasio is retained after the processing to create the ET tube coating, confirming the robust nature of this platform which could be potentially applied to any other AMP. Importantly, all ET tube coating conditions, as well as free peptide, were not cytotoxic toward human patient-derived laryngotracheal fibroblasts (FIG. 5C).

Overall, Lasio/PLGA coated ET tubes represent a clinically translatable technology that is fast and easy to produce and can elute AMPs predictably and continuously over the normal duration of chronically intubated patients. Additionally, the activity of the coated tubes can be easily modulated by replacing the broad-spectrum AMP Lasioglossin-III with other AMPs that exhibit selectivity toward a specific microbial target. For example, the ET tube coating could be loaded with the MADI, an AMP that selectively kills Mycobacterium species,¹⁴ which are overabundant in idiopathic SGS patients. 6 Future work in determining the baseline airway microbiome and microbiome in cases of laryngotracheal stenosis would allow us to target certain pathogens and prevent the inflammatory cascade. The inventors believe that with the ability to purposefully select an AMP/PLGA coated ET tube against certain microbes in the trachea and other organs could have significant implications in the prevention of specific diseases including, but not limited to, SGS.

In sum, the inventors demonstrated the design of a peptide-eluting ET tube based on a PLGA matrix that results in a uniform coating that continuously and linearly release Lasioglossin-111 in amounts sufficient to kill specific bacteria and polymicrobial microbiome cultures. In conclusion, the inventors offer an improved device to modulate the upper-airway microbiome and that could be deployed to prevent bacterial infections during intubation and help prevent subglottic stenosis and other upper airway diseases.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Jefferson N D, Cohen A P, Rutter M J, Subglottic stenosis, Semin Pedtatr Surg. 2016; 25(3)-138-143. doi:10.1053/j.sempedsurg.2016.02.006
2. Hillel A T, Karatayli-Ozgursoy S, Beake J R, et al. Voice quality in laryngotracheal stenosis. Ann Otol Rhino/Laryngol. 2015; 124(5):413-412. doi:10.1177.0003489414564249
3. Gelbard A, Francis D O, Sandulache V C, Simmons J C, Donovan D T, Oagkasawan J. Causes and consequences of adult laryngotracheal stenosis. Laryngoscope. 2015; 125(5):1137-1143, doi:10.1002/lary.24956
4. Feinstein A J, Goel A, Rogbavan G, et al. Endoscopic management of subglottic stenosis. JAMA Otolaryngof⋅Head Neck Surg 2017; 143(5):500-505. doi:10.1001/jamaoto.2016.4131
5. Axtell A L, Mathisen D J. Idiopathic subglottic stenosis: Techniques and results. Ann Cardiothorac Surg. 2018; 7(2):299-305. doi:10,2103 7/acs.2018.03.02
6. Gelbard A, Kataantonis N-G, Mizuta M, et at Molecular analysis of idiopathic stenosis far Mycobacterium species. Laryngoscope. 2017; 127(1):179-185. doi:10,1002/lary.26097
7. Hillel A T. Tang S S, Cados C, et al. Laryngotracheal Microbiota Adult Laryngotracheal Stenosis. mSphere. 2019; 4(3), doi:10.1128/mspheredirect.00211-19
8. Brogden K A. Antimicrobial peptides: Pore formers or metabolic inhibitors in bacteria?Nat Rev Microbial. 2005:3(3):238.250.
9. Fjell C D, Hiss J A, Hancock R E W, Schneider G. Designing antimicrobial peptides: Form follows function. Nat Rev Drug Discov. 2012; 11(1):37-51.
10. Lazzaro B P, Zasloff M, Rolff J. Antimicrovial peptides: Application informed by evolution. Science (80-). 2020; 368(6490):eaau5480.
11. Elliott A G, Huang J X, Neve S, et at. An amphipathic peptide with antibiotic activity against multidrug-resistant Gram-negative bacteria. Nat Commun. 2020:11(1):1-13. doi:10,10381s41467-020-16950-x
12. Rezat M S, Pourxmousa R Dadashzadeh R, Ahangarkani F. Multidrug resistance pattern of bacterial agents isolated from patient with chronic sinusitis. Casp J Intern Med. 2016; 7(2):114-119. /pmc/articles/PMC4913714/. Accessed Mar. 1, 2021.
13. Alves-Barroco C, Rivas-Garcia L,Fernades A R, Baptista P V. Tackling Multidrug Resistance in Streptococci—From Novel Biotherapeutic Strategies to Nanomedicines. Front Microbial. 2020; 11:2487. doi:10.3389fmicb.2020.579916
14. Simonson A W, Mongia A S, Aronson M R, et a. Pathogen-specific antimicrobials engineered de nova through membrane-protein biomimicry. Nat Biomed Eng. January 2021:1-14. doi:10.1038/s41551-020-00665-x
15. Torres M D T, Cao 3, Franco O L, Lu T K, de la Fueute-Nunez C. Synthetic Biology and Computer-Based Frameworks for Antimicrobial Peptide Discovery. ACS Nano. February 2021. doi:10.102/acsnano.OC09509
16. Kazetozadeh-Narbat M, Cheng H, Chabok R, et al. Strategies for amicrobial peptide coatings on medical devices: a review and regulatory science perspective. Crit Rev Biotechnol. 2021; 41(1).94-120. doi:10,1080/073885512020.1828810
17. Simonson A W, Aronson. MR, Medina S H, Medina S H. Supramolecular Peptide Assemblies as Antimicrobial Scaffolds. Molecules. 2020; 25(12):2751 doi:10.3390/molecules25122751
18. Tandel H, Florence K, Mira A. Protein and Peptide Delivery though Respiratory Pathway. In: Challenges in Delivery of Therapeutic Genomics and Proteomics. Elsevier Inc.; 2011:429-479. doi:10.1016/B978-0-12-38494-900009-8
19. Sharma A. Respiratory Distress. In: Nelson Pediatric Symptom-Based Diagnosis. Elsevier Inc.; 2018:39-60.el. doi:10.1016/B978-0-323-39956-2.00003-0
20. Oxcedit B, Pasic P, Sangwma P, et al Ekluaoun of the Novel Antimicrobial BCP3 in a Coating for Endotracheal Tubes. ACS Omega. 2020, 5(18):10288-10296. doi: 10.1021/acsomea.9b04178
21. Aiman A A, Gruber M, Martin P, et al. Local delivery of mometasone furoate from an eluting endotracheal tube. J Control Release 2018:272:54-61. doi:10.1016:j.jcoarel.2018.01.003
22. Hashemi M M, Rovig J, Bateman J, et al Preclinical testing of a broad-spectrum antimicrobial endotracheal tube coated with an innate immune synthetic mimic. J Antimicrob Chemother. 2018; 73(1):143-150. doi 10.1093; jac/dkx347
23. Seitz A P, Schumacher F, Baker J, et al. Sphingosin coating of plastic surfaces prevents ventilator-associated pneumonia. J Mal Med. 2019; 97(8): 1 195-1211. doi:10.1007/s00109-019-01800-1
24. Cefovsky V, Budesiasky M, Hovorka 0, et at Lasioglossins: Three Novel Antimicrobial Peptides rom the Venom of the Eusocial Bee Lasioglossum laticeps (Hymenoptera: Halictidae) ChemBioChem. 2009; 10(12):2089.2099. dci:10.1002/cbic.200900133
25. Bayer E A, Jordan J, Roy A, et al. Programmed platelet-derived growth factor-bb and bone morphogenetic protein-2 delivery tom hybrid calcium phosphate alginate scaffold. Tissue Eng—Part A. 2017; 23(23-24):1382-1393. doi:10.1089/ten.tea.2017.0027
26. Tian Y, He X, Torralba M, et al Using DGGE profiling to develop a novel culture medium suitable for oral microbial communities. Mal Oral Microbal. 2010, 25(5):357-367.doi:10.1111/j.2041-1014 2010.005.x
27. Aronson M R, Dahl E S, Halle J A, et al, Re-eugieering Antimicrobial Peptides into Oncolytics Targeting Drug-Resistant Ovarian Cancers. Cell Mal Bioeng. June 2020:1-15. doi:10.1007:s12195-020-00626-z
28. House J C, Noordzsj J P, Murgia B, Langmore S. Laryngeal injury from prolonged intubation: A prospective analysis of contributing factors. Laryngoscope. 2011; 121(3):596-600. doi 10.1002/lary.21403
29. Zur K B, Mandell D L, Gordon R E, Holzman I, Rothschild M A. Electron microscopic analysis of biofilm on endotracheal tubes removed from intubated neonates. Otolaryngol—Head Neck Surg. 2004; 130(4):407-414. doi:10.1016/j.otohns 2004.01.006
30. Glowacki A, Gottardi R, Yoshizawa S, et al. Strategies to Direct the Enrichment, Expansion, and Recruitment of Regulatory Cells for the Treatment of Disease. Ann Biomed Eng 2015; 43(3):593-602.doi:10.10071s10439-014-1125-2
31. Gotiardi R L, Alexander P, Bianconi P A, Little S R. Treating soft tissue via controlled drug release. March 2015.
32. Siegel S J, Weiser J N. Mechanisms of Bacterial Colonization of the Respiratory Tract. Ann Rev Microbial. 2015; 69(1):425-444. doi:10.1146/annrev-micro-091014-104209
33. Wang L min, Qiao X liang, Ai L, Zhai J jing Wang X xia. Isolation of antimicrobial resistant bacteria in upper respiratory tract infections of patients. J Biotech. 2016; 6(2) doi:10.1007/s13205-016-0473-z
34. Perkins S D, Woeltic K F, Angenent L T. Endotracheal tube biofilm inoculation of oral flora and subsequent colonization of opportunistic pathogens. Int J Med Micobial. 2010; 300(7):503-511. doi:10.1016/j.ijmm.2010.02.005

1. An airway intervention device where a surface of said device is coated with a material comprising one or more anti-microbial peptides (AMPs) and one or more hydrophobic and/or natural polymers.

2. The device of claim 1, wherein said device is a tracheal tube, a stent, a mask, a tracheostomy tube, a catheter, an oral retainer, a balloon, a patch, or a packing material.

3. The device of claim 1, wherein the polymer is poly-(lactic-co-glycolic acid), poly glycolic acid, poly lactic acid, poly (lactic-co-glycolic acid), or any combination thereof, or poly caprolactone, hydrogel, alginate, polyurethane, polyester, poly (ethylene terephthalate), poly anhydrides, poly orthoesters, poly beta-amino esters, chitosan, hyaluronic acid, cellulose, collagen, gelatin, silk fibroin, and/or cyclodextrin.

4. The device of claim 1, wherein the one or more AMPs is/are an anionic peptide, a linear cationic α-helical peptide, a cationic peptide enriched in one or more of proline, arginine, phenylalanine, glycine or tryptophan, or a anionic/cationic peptide containing cysteine with at least one intrapeptide disulfide bond, and optionally may contain one or more feature selected from a β-hairpin structure, a cyclic peptide structure, enrichment in any natural amino acid, a noncanonical amino acid, self-assembly, or D, L or D/L enantiomeric amino acids.

5. The device of claim 4, where the anionic peptide is a dermicidin or Maximin H5.

6. The device of claim 4, wherein the linear cationic α-helical peptide is a halictine, a citropin, an aurein, a temporin, a macropin, a cecropin, an andropin, a moricin, a ceratoxin, a melittin, a magainin, a dermaseptin, a bominin, brevinin-1, an esculentin, buforin II, a lassioglossin, CAP18 or LL37.

7. The device of claim 4, wherein the cationic peptide enriched in one or more of proline, arginine, phenylalanine, glycine or tryptophan is an abaecin, a drosocin, an apidaecin, a diptericin, an attacin, a prophenin, or an indolicidin.

8. The device of claim 4, wherein the anionic/cationic peptide containing cysteine with at least one intrapeptide disulfide bond is a brevenin, a protegrin, a tachyplesin, a defensin or a drosomycin.

9. The device of claim 1, wherein the coated material permits controlled release of said AMP, such as 1 ng/day to 200 μg/day.

10. The device of claim 1, wherein said material further comprising an anti-inflammatory agent, such as a steroid or an NSAID, or an additional molecule that impair a bacterial or viral agent, such as a protein that interferes with pathogen attachment, colonization, a protein that enhances immune clearance of said agent, or a conventional antibiotic, such as azithromycin, tobramycin, ciprofloxacin, erythromycin, and amoxicillin.

11. A method of preparing a coated airway intervention device comprising:
(a) providing an airway intervention device;
(b) immersing said device in an emulsion comprising one or more hydrophobic or natural polymers and one or more anti-microbial peptides (AMPs) to coat said device;
(c) repeating step (b) at least once, optionally twice; and
(d) drying said coated device.

12. The method of claim 11, wherein said device is a tracheal tube, a stent, a mask, a tracheostomy tube, a catheter, an oral retainer, a balloon, a patch, or a packing material.

13. The method of claim 11, wherein the polymer is poly-(lactic-co-glycolic acid), poly glycolic acid, poly lactic acid, poly (lactic-co-glycolic acid), or any combination thereof, or is poly caprolactone, hydrogel, alginate, polyurethane, polyester, poly (ethylene terephthalate), poly anhydrides, poly orthoesters, poly beta-amino esters, chitosan, hyaluronic acid, cellulose, collagen, gelatin, silk fibroin, and/or cyclodextrin.

14. The device of claim 11, wherein the one or more AMPs is/are an anionic peptide, a linear cationic α-helical peptide, a cationic peptide enriched in one or more of proline, arginine, phenylalanine, glycine or tryptophan, or a anionic/cationic peptide containing cysteine with at least one intrapeptide disulfide bond, and optionally may contain one or more feature selected from a β-hairpin structure, a cyclic peptide structure, enrichment in any natural amino acid, a noncanonical amino acid, self-assembly, or D, L or D/L enantiomeric amino acids.

15. The method of claim 14, where the anionic peptide is a dermicidin or Maximin H5.

16. The method of claim 14, wherein the linear cationic α-helical peptide is a halictine, a citropin, an aurein, a temporin, a macropin, a cecropin, an andropin, a moricin, a ceratoxin, a melittin, a magainin, a dermaseptin, a bominin, brevinin-1, an esculentin, buforin II, a lassioglossin, CAP18 or LL37.

17. The method of claim 14, wherein the cationic peptide enriched in one or more of proline, arginine, phenylalanine, glycine or tryptophan is an abaecin, a drosocin, an apidaecin, a diptericin, an attacin, a prophenin, or an indolicidin.

18. The method of claim 14, wherein the anionic/cationic peptide containing cysteine with at least one intrapeptide disulfide bond is a brevenin, a protegrin, a tachyplesin, a defensin or a drosomycin.

19. The method of claim 11, wherein the coated material permits controlled release of said AMP, such as 1 ng/day to 200 μg/day.

20. The method of claim 11, wherein said material further comprising an anti-inflammatory agent, such as a steroid or an NSAID, or an additional molecule that impair a bacterial or viral agent, such as a protein that interferes with pathogen attachment, colonization, a protein that enhances immune clearance of said agent, or a conventional antibiotic, such as azithromycin, tobramycin, ciprofloxacin, erythromycin, and amoxicillin.

21. A method of providing airway intervention to a subject comprising inserting an airway intervention device of claim 1 to subject.

22. The method of claim 21, wherein said intervention is performed for less than 1 day.

23. The method of claim 21, wherein said intervention is performed for more than 1 day, such as for a week, two weeks, three weeks, four weeks, five weeks, six weeks, seven weeks, eight weeks, nine weeks or 10 weeks.

24. A kit comprising an airway intervention device of claim 1, or the individual components for making such a coated airway intervention device, in sterile packaging.

25. The kit of claim 24, further comprising instructions for use of said device.

26. A method of reducing the incidence of subglottic stenosis, pneumonia (VAP), laryngeal infection, post-operative dressing/packing-induced infection, upper airway infection, rhinosinusitis, choanal atresia, or vocal fold injury and paralysis in subjects receiving airway intervention comprising providing airway intervention with an airway intervention device of claim 1.
